(19) United States
(12) Patent Application Publication
(10) Pub. No.: US 2011/0028821 A1
(43) Pub. Date: Feb. 3, 2011

(54) Electrocardiographic Monitoring System and Method Using Orthogonal Electrode Pattern

(75) Inventors: Bosko Bojovic, Belgrade (RS); Ljupco Hadievski, Belgrade (RS); Dorin Panescu, San Jose, CA (US)

Correspondence Address: Nixon Peabody LLP, P.O. Box 60610, Palo Alto, CA, 94306, US

(73) Assignee: NewCardio, Inc., Santa Clara, CA (US)

(21) Appl. No.: 12/534,064

(22) Filed: Jul. 31, 2009

(51) Int. Cl. A61B 5/0402

(52) U.S. Cl. 600/382; 600/509

(57) ABSTRACT

A system for monitoring a cardiac condition of a patient includes a diagnostic center configured to construct a 12-lead ECG of a patient using a special ECG signals numbering less than twelve by combining the special ECG signals with a transformation matrix, and a wearable device configured to generate the special ECG signals and including. The wearable device includes a belt having one or more belt electrodes, a waistband having one or more waistband electrodes, the belt and waistband electrodes configured to contact the skin of the patient and obtain electrical signals therefrom, and a host unit in electrical communication with the belt and waistband electrodes, the host unit including circuitry for generating the special ECG signals from one or more of the acquired electrical signals and circuitry for special ECG signals to a location remote from the wearable device.

### CROSS-REFERENCE TO RELATED APPLICATIONS

This application may be considered related to U.S. patent application Ser. No. 10/568,868 filed Feb. 21, 2006, which claims priority to the International Application No. PCT/YU04/00020 filed Aug. 19, 2004.

### TECHNICAL FIELD

The present invention relates to the field of medical electronics, more precisely to the field of instruments for measuring and recording bioelectric signals, such as electrocardiographs (ECGs).

### BACKGROUND

The concept of a system for urgent cardiac diagnostics, in which measurements for ECG determination are obtained from the patient and sent to a remote diagnostic center for possible intervention, is known. Such systems rely on measurements taken by the patient, and then, on the basis of these and a conversation with the patient, a cardiologist at a remote location can decide: a) whether an urgent intervention is needed, b) whether the intervention can be performed by the patient himself, or c) whether the patient's state requires urgent medical intervention, and acts accordingly. It is very important that the most critical period, from the occurrence of the first symptoms until the medical treatment, be minimized (Lenfant C. et al.: Considerations for a national heart attack alert program, Clin. Cardiol. 1990 Aug.; 13 (8 Suppl 8): VIII9-11). There is a number of patents and products which, within the concept of urgent cardiological diagnostics, offer different solutions for recording and transmitting the ECG signal: U.S. Pat. No. 4,889,134 Greenwold, et al., 1989; U.S. Pat. No. 5,226,431 Bible, et al. 1993; U.S. Pat. No. 5,321,618 Gessman, 1994; U.S. Pat. No. 5,966,692 Langer, et al., 1999; PCT WO 01/70105 A2, B. Bojovie 2001; “Instant Memory Recorder” of the company TELESCAN MEDICAL SYSTEMS (TELESCAN MEDICAL SYSTEMS 26424 Table Meadow Road, Auburn, Calif. 9560); “CardioCall Event Recorder” by REYNOLDS MEDICAL (REYNOLDS MEDICAL LTD, John Tate Road, Hertford SG13 7NW United Kingdom) and “Heartwiev P-12” by AEROTEL (AEROTEL LTD. 5 Hazoref st. Holon 58856 Israel). The solutions can be divided into three groups:

1) The first group comprises solutions for sending the recording of one or two standard ECG leads. The mobile recorders of this group can be very small and with integrated electrodes (no cables are needed), which is the advantage of the group. The recording is performed by simple holding of the device on the patient's chest or by positioning the fingers on the integrated electrodes. This is a quick and simple way for a patient to record one or two leads of his ECG. However, recording one or two ECG signals limits the application of these devices to the patients with rhythm disorders, which is about 20% of the patient population with heart diseases. Typical device of this group is “CardioCall Event Recorder” by REYNOLDS MEDICAL.

2) The second group consists of solutions that enable direct recording and transmission of standard 12-lead ECG, thus including their application to the patients with the diagnoses of coronary artery diseases. Namely, in such patients, the complete standard 12-lead ECG is necessary for urgent diagnostics. Some of these devices are equipped with the full set of electrodes and cables for recording all 12 standard ECG leads (usually 10 electrodes, that is cables), which a patient himself attaches onto his body during recording. The typical representative of this group is “12 Lead Memory ECG Recorder” by TELESCAN MEDICAL SYSTEMS. The other method is the use of a reduced number of electrodes that are moved during the recording. For example, if four electrodes are used, three are positioned at the locations of standard ECG leads I, II, and III (arms and legs of the patient), while the fourth electrode has to be moved during recording to each of the six chest positions for recording chest leads V1-V6 (U.S. Pat. No. 4,889,134, Greenwold et al., 1989). The method that uses three cable connected electrodes and four button-shaped integrated electrodes can be found in the device “Heartwiev P-12” by AEROTEL. The recording of 12 leads is performed in three steps: leads DI, D2, D3, aVR, aVL, aVF, V1, and V2 are recorded in the first step, V3 and V4 in the second, and V5 and V6 in the third step. The common disadvantage of the whole group is rather complicated and long-lasting recording procedure, which makes them very inconvenient for self-application, especially for the patients suffering a heart attack. Significant errors are, however, possible, due to imprecise positioning of the electrodes.

3) The third group includes the solutions in which a reduced number of special leads is recorded, and later, on the basis of this recording, all 12 standard ECG leads are reconstructed computationally. The method for the reconstruction of 12 standard ECG leads and/or x,y,z leads of a vectorcardiogram based on the recorded special leads obtained with four electrodes is explained in U.S. Pat. No. 4,850,370, G. E. Dower 1989. The method is based on the dipole approximation of the electrical heart activity and uses the universal tranformation matrix T, with dimensions 3×12, and with the matrix coefficients determined experimentally. A similar solution is given in EASI system method (http://www.healthcare.philips.com/main/products/patient_monitoring/products/ecg/index.wpd).

The conventional ECG leads V (I, II, III, aVR, aVL, aVF, V₁, V₂, V₃, V₄, V₅, V₆) are obtained by multiplying the transformation matrix T with the recorded signals at the special leads Vₛ(Vₛ₁, Vₛ₂, Vₛ₃). The universal transformation matrix for all patients does not contain information about individual characteristics of a patient, which results in major errors in the reconstruction of the standard ECG lead signals. In this setup, the quality of signal reconstruction is highly dependent on the proper positioning of special leads electrodes.

An improvement of this method by introducing the individual transformation matrix is given in the paper by Scherer, J. A. et al., Journal of Electrocardiology, v 22 Suppl, pp. 128, 1989, and applied in the U.S. Pat. No. 5,058,598 (J. M. Niklas et al., 1993), wherein the implementation of the individual transformation matrix for each patient, with the segment calculation of the transformation matrix coefficients, was suggested (ECG signal is divided into segments and the coefficients for each segment are calculated individually). The reconstruction of the standard ECG lead signals by the individual transformation matrix means that it is necessary to perform the basic (calibrating) recording for each patient, which will be used for the matrix coefficient calculation. The errors in this approach are significantly reduced compared to the method using the universal transformation matrix. The major drawback of both of these methods is the need to use cables for recording with the suggested arrangement of electrodes, which is very inconvenient for self-application, especially in patients suffering a heart attack. The method in which the reconstruction of standard ECG leads is also done with the individual transformation matrix (Scherer, J. A. et al., Journal of Electrocardiology, v 22 Suppl, pp. 128, 1989), but with the mobile ECG device with integrated electrodes, i.e. with no cables used, is presented in the patent PCT WO 01/70105 A2, B. Bojovic 2001. The device enables quick and easy recording of the special ECG leads and reconstruction of all 12 standard ECG leads with the individual transformation matrix. However, the limitations in the arrangement of the electrodes, due to the use of the integrated ones, disable the optimal arrangement of electrodes on the patient's body, which can result in errors in the signal reconstruction.

An additional problem present in all three groups is the occurrence of the base line wandering of the ECG signal during recording. The effect is especially undesirable for the third group of the devices because the base line wandering during the recording of special leads brings about major diagnostic errors in the procedure of the reconstruction of 12 standard ECG leads.

### OVERVIEW

As described herein, a wearable device is configured to generate special ECG signals for constructing a 12-lead ECG, the special ECG signals numbering less than twelve and being combinable with a calibration matrix in order to construct the 12-lead ECG. The wearable device includes a belt having one or more belt electrodes, a waistband having one or more waistband electrodes, the belt and waistband electrodes configured to contact the skin of a wearer and obtain electrical signals therefrom, and a host unit in electrical communication with the belt and waistband electrodes, the host unit including circuitry for generating the special ECG signals from one or more of the acquired electrical signals and circuitry for transmitting information based on the special ECG signals to a location remote from the wearable device.

Also as described herein is a system for monitoring a cardiac condition of a patient that includes a diagnostic center configured to construct a 12-lead ECG of a patient using a special ECG signals numbering less than twelve by combining the special ECG signals with a transformation matrix, and a wearable device configured to generate the special ECG signal. The wearable device includes a belt having one or more belt electrodes, a waistband having one or more waistband electrodes, the belt and waistband electrodes configured to contact the skin of the patient and obtain electrical signals therefrom, and a host unit in electrical communication with the belt and waistband electrodes, the host unit including circuitry for generating the special ECG signals from one or more of the acquired electrical signals and circuitry for transmitting information based on the special ECG signals to a location remote from the wearable device.

Also described herein is a method for generating a 12-lead ECG of a patient. The method includes using belt-mounted electrodes to obtain electrical signals from the patient, using waistband-mounted electrodes to obtain electrical signals from the patient, using a host unit to generate special ECG signals from the electrical signals obtained from the belt-mounted and waistband-mounted electrodes, and to transmit information based on the special ECG signals to a diagnostic center, the special ECG signals numbering less than 12, and constructing a 12-lead ECG by combining the special ECG signals with a transformation matrix.

Also described herein is a wearable device configured to generate special ECG signals for constructing a 12-lead ECG, the special ECG signals numbering less than twelve and being combinable with a calibration matrix in order to construct the 12-lead ECG, the wearable device including: a first wearable component having one or more electrodes configured to contact the skin of a wearer and obtain electrical signals therefrom; and a host unit in electrical communication with the first wearable component, the host unit including circuitry for generating the special ECG signals from one or more of the acquired electrical signals and circuitry for transmitting information based on the special ECG signals to a location remote from the wearable device.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

In the drawings:

FIG. 1 is a diagram of a bedside cardiac monitoring system;

FIG. 2 is a diagram of a wearable device for use with a bedside cardiac monitoring system;

FIG. 2A is a diagram showing an electrode configuration of a belt of wearable device;

FIG. 2B is a diagram showing a host unit-mounted electrode;

FIG. 2C is a diagram of a wearable device having a wearable component in the form of a shirt or blouse;

FIG. 2D is a schematic diagram showing a wearable device having wireless internal communication;

FIG. 2E is a block diagram showing details of an electrode transmitter module;

FIG. 2F is a block diagram showing details of an electrode receiver module;

FIG. 3 is a block diagram illustrating circuitry of a host unit that uses a common electrode;

FIG. 3A is a block diagram illustrating an alternative circuit arrangement that does not use a common electrode; and

FIG. 4 is a flow diagram of a process for calibrating a bedside cardiac monitoring system and monitoring information therefrom.

### DESCRIPTION OF EXAMPLE EMBODIMENTS

Example embodiments are described herein in the context of a bedside cardiac monitoring system and method using an orthogonal electrode pattern. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used to the extent possible throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

A system and method are described herein for reliably and comfortably monitoring patient ECG over a period of interest, and is particularly useful for patients who have undergone cardiac related procedures such as angioplasty, stenting, bypass surgery and the like and need to be monitored for a period of time following the procedure, or for patients who are known to be at risk for cardiac events such as heart attacks and need to be continuously monitored as part of bedside care.

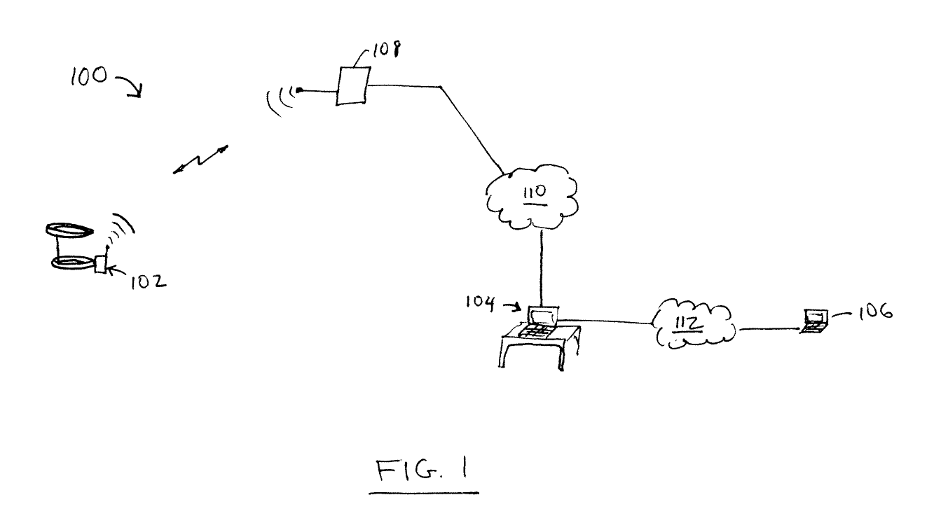

A system such as that shown in FIG. 1 can be used for the above procedure, and performs cordless/wireless recording, transmission, and processing of three special ECG leads. The measurements obtained are delivered, preferably wirelessly, to a diagnostic center where they are reconstructed, using a transformation matrix, which can be an individual transformation matrix specific to the patient, to produce the patient's ECG in real-time for monitoring by automated equipment and/or staff at the diagnostic center or at a remote location in communication with the diagnostic center. The system, shown generally at 100, includes a wearable device 102 that extracts three special ECG signals from the patient, who can wear the device and sleep in it comfortably for an extended period of time, such as several days, for constant monitoring. The wearable device 102 wirelessly transmits the extracted signals, or derivation signals and information based thereon, to diagnostic center 104. Transmission can be in real time, or the information can be stored in the wearable device 102 and transmitted in bursts at designated intervals (once each hour, etc.), or it can be retained in the wearable device for subsequent downloading at the diagnostic center 104, in the manner of a Halter-type device, for example through a USB cable or other connection, or through Bluetooth or other wireless expedient. The transmitted or stored information can be raw data, or it can be data that has been partially or completely processed at the wearable device, as explained in greater detail below.

In a hospital setting, the patient (not shown) wearing the wearable device 102 can be in one location, such as a private or shared room, while the diagnostic center 104 can be at a different location or room. The ECG generated at the diagnostic center 104 can be displayed and monitored there, or at a remote location 106 on or off the hospital premises and in communication with the diagnostic center. It will be appreciated that while a single wearable device 102 is depicted, it is possible for diagnostic center 104 to be in communication with multiple such devices so that a plurality of patients can be monitored simultaneously by the single diagnostic center. Of course in that case, when individualized transformation matrices are used, the diagnostic center 104 would contain multiple individualized transformation matrices each associated with a specific patient so that the individual patient's ECG can be reconstructed.

Also shown in FIG. 1 is a wireless access point 108 with which diagnostic center 104 can communicate with wearable device 102. One or more such access points may be provided to improve access, especially when multiple patients using multiple wearable devices and located at different regions in a care facility such as a hospital are involved. Communication between wearable device 102 and diagnostic center 104, and between the diagnostic center and the remote location 106, may be by way of one or more networks, such as local area network (LAN) 110, wide area networks (WANs), the Internet 112, and so forth, and paths between devices can be wireless or wired, or a combination of wireless and wired, and can pass through no networks or through one or more different networks. Those of ordinary skill in the art will recognize that many network configurations are possible to facilitate communication between wearable device 102 and diagnostic center 104, and these may be functions of the distances between the devices, the complexity of the system, the number of wearable devices 102/patients being monitored, the number of diagnostic centers 104 involved as a multiplicity of these are also contemplated, the number of remote locations 106, and so on.

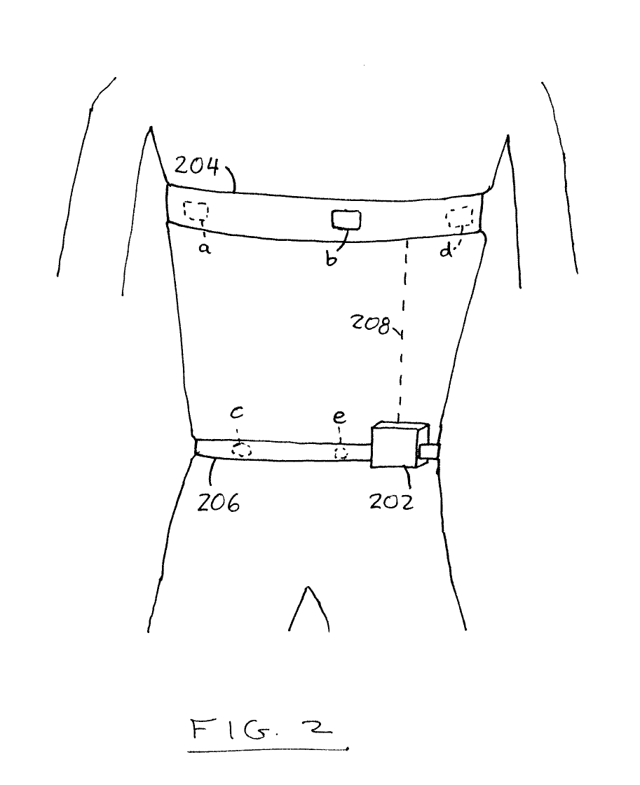

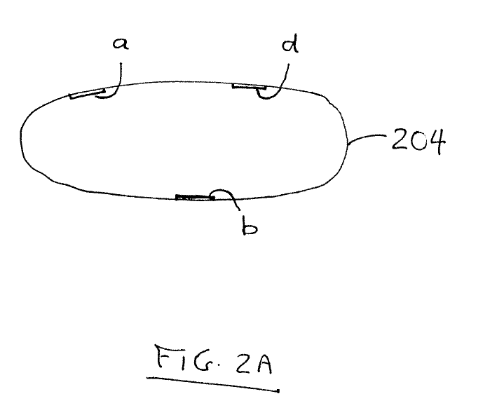

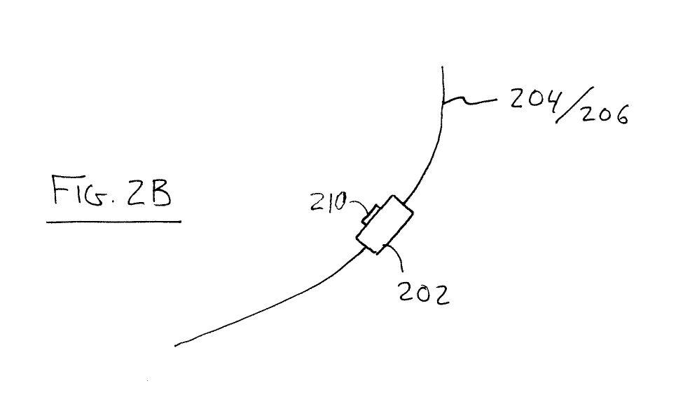

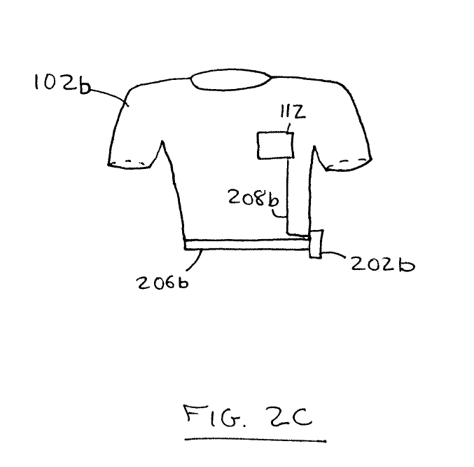

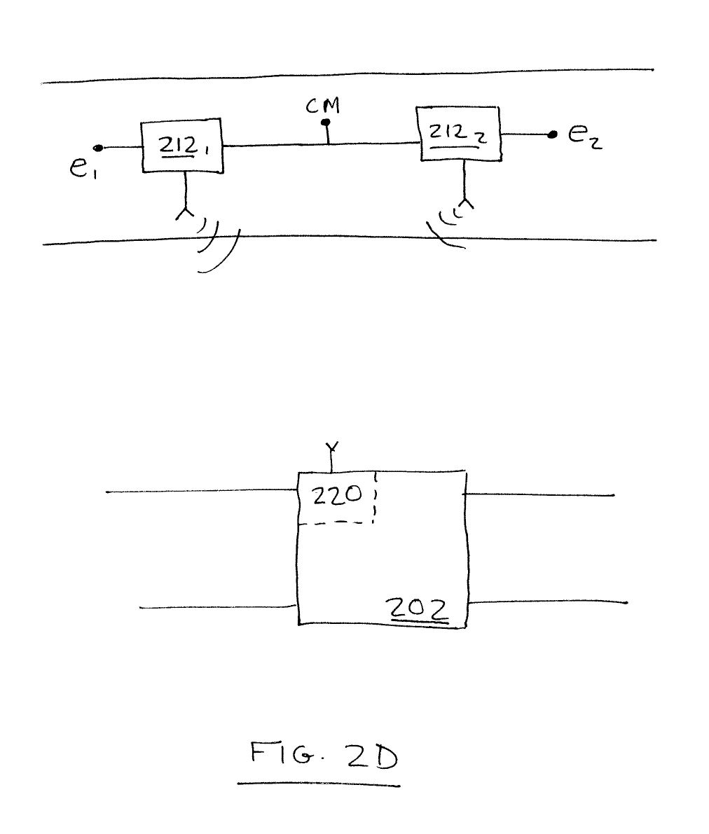

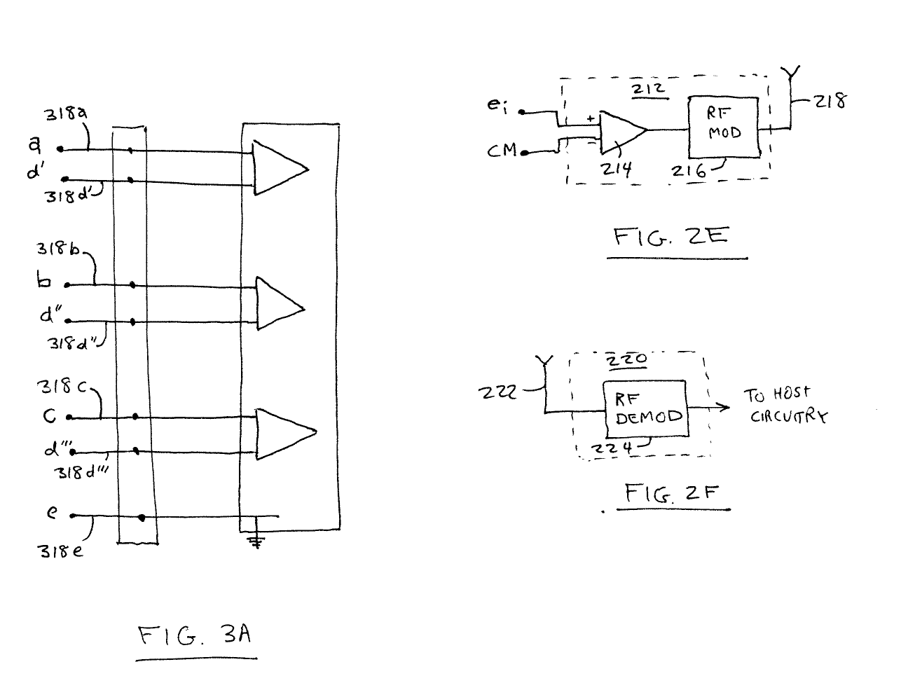

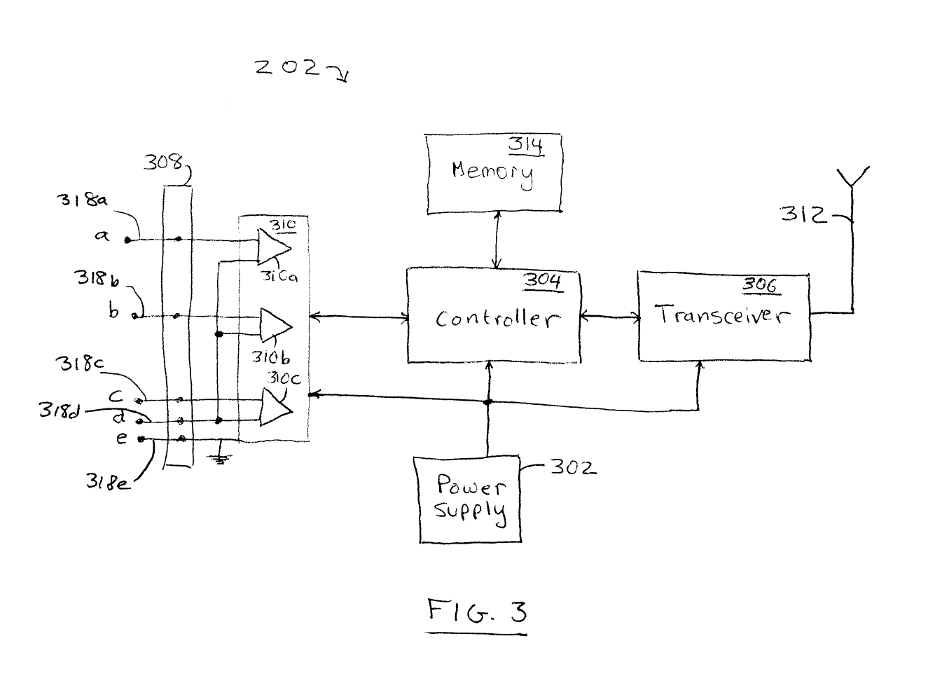

FIG. 2 shows a more detailed view of wearable device 102, which is generally in the form of a host unit 202 housing the main electrical components (detailed in FIG. 3) and coupled to wearable components in the form of a belt 204 and a waistband 206. The belt, waistband and host unit together present a set of electrodes (a-e) for contact with the skin of the patient, and are distributed on the belt (belt electrodes) and waistband (waistband electrodes), and possibly the host unit (host unit electrodes), in accordance with a non-coplanar arrangement for establishing an orthogonal electrode pattern as explained below. In the arrangement of FIG. 2, the host unit 202 is physically coupled to the waistband 206, but this is not mandatory and the host unit can instead be coupled to the belt 204, or can be independent of the two. In any of these cases the host unit 202 is in electrical communication with the belt electrodes and waistband electrodes such that signals from the patient acquired by these electrodes are delivered to the host unit. Depending on the exact configuration and the contemplated electrode distribution, a wired or wireless connection 208 can be provided between the host unit 202 and belt 204, and possibly an additional wired or wireless connection (not shown) can be provided between the host unit and the waistband 206.

Different schemes for the electrodes (a-e) can be utilized, with the scheme shown in FIGS. 2 and 2A serving as merely one example. Importantly, an orthogonal electrode pattern is established, using at least four electrodes from which three special leads are derived. These at least four electrodes should be placed in a non-coplanar arrangement. Belt 204 is intended to be worn around the chest of the patient and as illustrated contains belt electrodes a, b, and d that contact the skin of the patient in the vicinity of his/her chest, upper waist and/or back. The electrodes a, b and d housed on waistband 206 are set about 120° apart in this example. Waistband 206 is intended to be worn around the lower waist of the patient and contains waistband electrodes c and e that contact the skin of the patient's lower waist. Other arrangements, including an opposite arrangement in which three electrodes are housed on belt 204 while two are housed waistband 206 are also contemplated. Also, while in this illustrative embodiment none of the electrodes are disposed on the host unit 202, it is possible to house one or more electrodes on the host unit 202 and mount the host unit to the waistband (or belt) such that these one or more host unit electrodes contact the skin of the patient. Such an example is shown in FIG. 2B, in which a host unit electrode 210 is shown disposed on the interior surface of host unit 202, which can be mounted on either belt 204 or waistband 206. Those of ordinary skill will recognize that other non-coplanar electrode arrangements are possible, and the invention is not limited to the specific example given herein.

It should be noted that while the wearable components of the wearable device 102 are in the form of a “belt” and a “waistband,” other expedients such as harnesses, bands and straps can be used in lieu of or in conjunction with one or both the belt and waistband, and can be associated with parts of the body of the patient other than the waist and chest. For example, either or both the belt and waistband can be replaced with patches that abut the skin of the patient, bringing it into contact with electrodes disposed on the patches. Such patches can be adhered to the skin using an appropriate adhesive, or they can be sewn or otherwise affixed to the interior of a special garment worn by the patient, or they can be strapped to the patient's body through any suitable means. FIG. 2C is directed to a garment arrangement, and shows a wearable device in which the wearable component is in the form of a shirt or blouse 102b having a patch 112 housing one or more patch electrodes (not shown), a waistband 206b housing one or more waistband electrodes, and a host unit 202b communicating with the patch and waistband electrodes, through an illustrative wired connection 208b, and transmitting signals therefrom to a diagnostic center (not shown).

All the electrodes (a-e) are connected to host unit 202 and provide electrical signals derived from the body of the patient to the host unit. Electrodes a-c provide special lead signals, electrode d provides a common signal, and electrode e provides a ground signal for the ECG reconstruction procedure as further explained below. The common signal from electrode d is used to efficiently provide a common reference point against which the potentials at electrodes a, b and c are measured. Alternatively, each of the electrodes a, b and c can be associated with its own reference point against which the potential is determined.

As mentioned above, some or all of the electrodes a-e can communicate wirelessly with the host unit. A general schematic of such wireless communication between two electrodes in this example and the host unit is shown in FIG. 2E. The electrodes e₁ and e₂ are each shown to be associated with a dedicated electrode transmitter module 212₁ and 212₂, although it is contemplated that the transmitter modules can be shared among two or more electrodes. A common electrode CM, for providing a reference signal, is also shown, coupled to the transmitter modules 212₁ and 212₂. As discussed above, a reference electrode can be provided for each of the electrodes individually, rather than using a common electrode to provide the reference for multiple electrodes. Details of the transmitter modules 212₁ and 212₂ are shown in FIG. 2F. Specifically, the electrode signal eᵢ is provided as a first input to a differential amplifier 214, and the common (or dedicated) reference electrode is provided as the second input. The output of the differential amplifier is provided to an RF (radio frequency) modulator 216 for transmission by way of an antenna 218.

The signals from transmitter modules 212₁ and 212₂ are received by a counterpart electrode receiver module 220 at the host unit 202. The receiver module 220 includes an antenna 222 and an RF demodulator 224. It is also contemplated that some or all the circuitry and components of transceiver 306, antenna 312 and controller, discussed in detail below, can be used to receive and process the signals from the wireless electrodes eᵢ in lieu of or in addition to the circuits and components of receiver module 220.

Details of host unit 202/202b are shown schematically in FIG. 3. These include power supply 302, controller 304, wireless transceiver 306, electrode interface 308, amplifier module 310 containing amplifiers 310a-310c, antenna (internal or external) 312, and memory 314. Electrode interface 308 receives electrical signals from leads 318a-318e coupled respectively to electrodes (a-e) (FIG. 2) and couples these electrically into the host unit as shown. Specifically, special leads 318a-318c are connected as inputs to corresponding amplifiers 310a-310c, common lead 318d is connected commonly to all three amplifiers 310a-310c as a reference input for special leads 318a-318c, and lead 318e is connected to ground for the three amplifiers. In the alternative embodiment mentioned above, three leads 318d′, 318d″ and 318d′″ connected respectively to electrodes d′, d″ and d′″ which are associated respectively with special electrodes a, b and c, can be used in lieu of common lead 318d. Such a configuration is illustrated in FIG. 3B. Electrode e connected to lead 318e is optional and provides improvements in noise rejection, serving to better equalize the patient potential to that of circuitry involved. As such, the minimum number of electrodes is four—a-c to provide the special leads, and d to operate as the common point of these. Electrode e is optional and serves to improve performance when needed.

The amplifiers 310a-310c amplify the signals from special leads 318a-318c and pass them to controller 304, which is optional and which can be used to provide management and control functions for the other components of the host unit 202 and wearable device 102. For instance, the operation of the amplifier module 310 can be monitored using controller 304 and feedback to the wearer or caretaker indicative of proper operation can be provided. As an example, the controller 304 can check for appropriate voltage levels received from the amplifiers, and if these are below predetermined thresholds, an indication that a lead is not properly positioned on the body of the patient can be provided, in the form of an acoustic tone or flashing LED (not shown), for instance. Conversely, proper connection and operation can be indicated by a different tone or an uninterrupted LED emission, or other indication. These indications can be provided at the wearable device 102, and/or at the diagnostic center 104 with which it is in communication. The indications can also be provided to guide the patient and/or caretaker during the calibration process detailed below, to for instance indicate successful or unsuccessful calibration, recording in-progress, and so on.

Other functions of the controller 304 can be to condition the signals received from the amplifier module 310 for transmission by transceiver 306 and antenna 312. Conditioning may include appropriately modulating a carrier wave for RF transmission, in accordance with any known protocol. Other components to facilitate transmission can be used, such as a modem, as is known, and any of myriad types of wireless or wired schemes for communication between wearable device 102 and diagnostic center 104 may be employed. Moreover, two-way communication is contemplated, such that antenna 312 and transceiver 306 can be configured to receive signals from diagnostic center 104 and/or other devices to pass on to controller 304. These signals can be for performance of a handshaking procedure for proper connection, an authentication procedure, or they can be command signals for controller 304, for example to recalibrate, or to provide a failure signal or indication at the wearable device 102.

The signals from amplifier module 310 can also be stored for subsequent downloading, and memory 314 is provided for this purpose. Memory 314 is preferably a persistent type device, such that information remains stored even after power-down. Power to the various components is provided by power supply 302, which can take the form of a rechargeable or disposable battery pack.

Using the above arrangement, it is possible to precisely reconstruct all 12 signals of standard ECG leads with only signals from the three special leads 201a-201c. This is performed by combining the special leads signals by a pre-generated transformation matrix, preferably one that is individualized for the particular patient. The reconstruction is conducted in real time, preferably at the diagnostic center 104, which contains computing resources that include hardware and/or software to perform the combining, generate the ECG therefrom, display it to the operator, and possibly conduct intelligent automated monitoring and signal any alarm conditions.

As explained above, the information obtained from the electrodes e-c can be transmitted to diagnostic center 204 in real time, or it can be stored, for example in memory 314, for subsequent transmission, at prescribed intervals or in a single burst. Alternatively, the information can be retained in the memory 314 for subsequent downloading at the diagnostic center, using a dedicated connection such as a cable, cradle, or wirelessly (BlueTooth™, etc.). Since the data used is from three special leads (and reference and/or ground), rather than the conventional 12 leads, the amount of information that needs to be stored is reduced and the memory requirements are similarly reduced. The information itself can be in raw form, or it can have undergone partial or complete processing in the host unit 202 to render an ECG for presentation to the caregiver or physician. Processed data is derived by combining the raw data with the personalized or general transformation matrix, for instance, which in this example would take place in the wearable device itself, and specifically in the host unit thereof. Alternatively, the 12-lead reconstruction could take place at the diagnostic center.

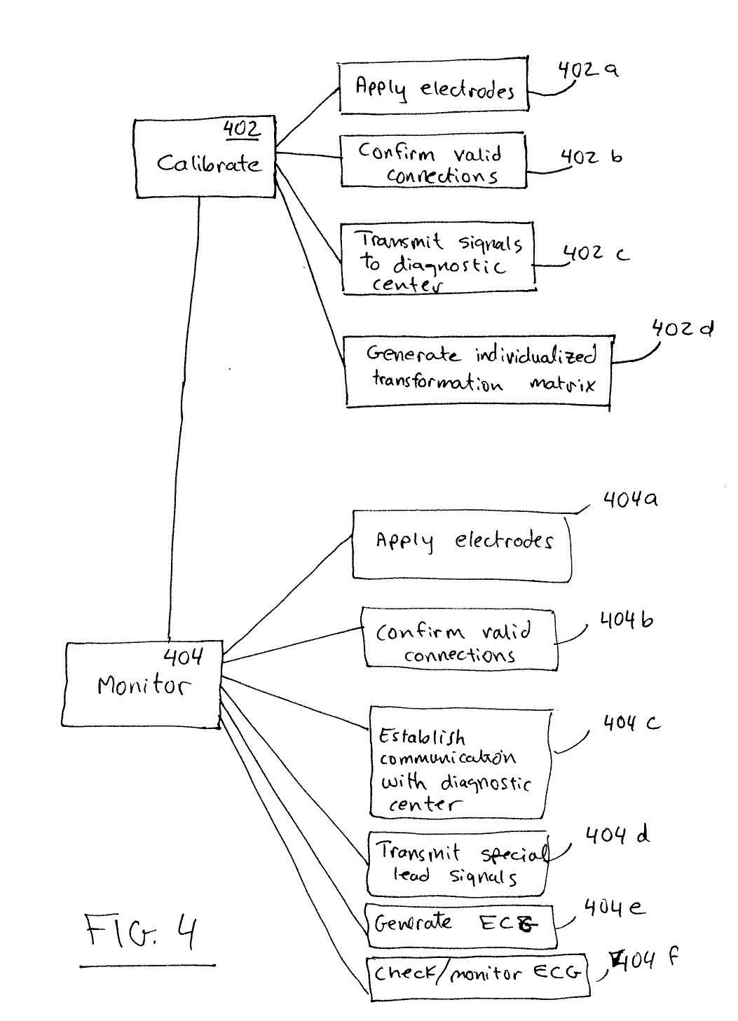

The process for monitoring a patient with the system 100 is explained with reference to FIG. 4. Initially, the individualized transformation matrix is calculated during a calibration step 402 in which a standard 12-lead ECG measurement is taken, using the wearable device 102 and a conventional ECG device having 12 actual leads, provided that this arrangement enables simultaneous recording of 3 special leads and conventional 12 leads. At 402a, the measuring device is fitted to the patient; at 402b proper connections are ascertained; at 402c signals of 3 special leads and conventional 12 leads are transmitted to diagnostic center; and at 402d the individualized transformation matrix is generated, using known techniques. In some situations a general transformation matrix may be used, and step 402 omitted.

Actual monitoring occurs after the calibration step, and is illustrated at 404 in FIG. 4. This includes the patient wearing wearable device 102, with belt 204 around the patient's chest and waistband 206 around the patient's lower waist, such that electrodes a-e come into contact with the patient's skin. Monitoring begins at 404a with fitting the patient with the wearable device 102. At 404b, confirmation of proper electrode-skin contact is performed. At 404c, a connection (wired or wireless) between wearable device 102 and diagnostic center 104 at 404c is established. At 404d, the three special lead signals are transmitted from wearable device 102 to the diagnostic center 104 over the established connection. At 404e, the 12-lead ECG of the patient is generated by combining the special lead signals with the individualized transformation matrix of the patient. At 404f, the ECG is checked for alarm conditions, which can be performed automatically or manually if the ECG is displayed. It will be appreciated that the step order disclosed above need not be strictly adhered to. For instance, establishment of communication between with diagnostic center 104 at step 404c can precede applying electrodes step 404a and/or confirming valid connections step 404b.

The accuracy in the reconstruction of 12 standard ECG leads using the recordings of three special leads is achieved using the arrangement of integrated electrodes as described herein. The reconstruction algorithm is based on the assumption that diffused electrical activity of the heart muscle can be approximated by a time-changing electrical dipole (heart dipole) immersed in a low conducting environment. Heart dipole is a vector defined by three non-coplanar projections, so that it can be determined on the basis of recording of electric potential in at least four points that correspond to three non-coplanar directions—that is, three ECG leads not lying on the same plane, with the fourth providing a reference, that may be common to all three (or it may be in the form of a separate electrode associated with each of the three special leads). Once the heart vector is determined, it is possible to calculate the electric potentials in any point, meaning the 12 standard ECG leads as well. The calculation of heart dipole is not necessary; the direct connection between the recorded special leads and standard ECG leads can be established instead, so that standard ECG leads are obtained as linear combinations of the recorded special leads and coefficients by which the transformation matrix is defined. However, direct application of this approach is facilitated by a detailed analysis of the error sources and an attempt to reduce them. Based on this analysis, it has been shown that there are two dominant error sources that should be taken into consideration.

### a) Model Errors

The system of reconstruction of the standard ECG leads on the basis of recording of three special leads is based on the dipole representation of heart electrical activity. However, the heart dipole is only the first term in the multipole expansion of diffused heart electrical activity and this approximation is valid only for recording points at the sufficient distance from the heart. In the points near the heart, the potential is significantly affected by the non-dipole content created due to the presence of higher order terms in multipole expansion.

### b) Transformation Matrix Calculation Errors

Practical calculation of transformation matrix T is conducted by the simultaneous recording of 12 standard ECG leads V(D₁, D₂, D₃, aVR, aVL, aVF, V₁, V₂, V₃, V₄, V₅, V₆) and three special leads Vₛ(Vₛ₁, Vₛ₂, Vₛ₃), followed by numerical solving of the equation V=T·Vₛ, by the least-squares method. The errors in recording the electric potentials introduce the errors in the calculation of transformation matrix coefficients. The analysis has shown that the errors can be minimized if the vectors of special leads recording points are orthogonal.

Finally, having in mind the model errors (a) and the transformation matrix calculation errors (b), two requirements are imposed concerning the arrangement of the electrodes for special leads recording, in order to minimize the total error. The first one is to position the electrodes of the special leads as far as possible from the heart; the second one is to arrange the electrodes in such a way that the vectors of recording points' positions are close to orthogonal as much as possible. By arranging the electrode positions as described herein, and choosing the common point, the optimal minimization of the model errors (a) and transformation matrix calculation errors (b) can be achieved. It should be noted that in this arrangement, the accurate positioning of the special leads is not critically important, provided that the initial positioning used in calibrations are maintained during monitoring. Furthermore, if the arrangement is to be re-applied by the patient, the application is more easy to be correctly repeated by the patient, compared to prior art.

An additional problem in signal recording of special as well as of standard ECG leads is the effect of the base line wandering of the recorded signals. The problem occurs during the recording of ECG signals with all kinds of ECG devices, but is more prominent with mobile ECG devices due to the more difficult recording conditions. When systems which obtain standard ECG leads by the reconstruction of recorded special leads are concerned, the elimination of the base line wandering problem during recording of special leads is important for the proper functioning of the system. The controller 304 can be used to establish control of the base line wandering during recording of special leads by managing the process of recording automatically. From the moment of putting the device into the recording position until the moment when the base line of a signal fits into the previously specified range, a characteristic sound signal can be emitted. During the next period defined by the signal relaxation time, another characteristic sound signal can be being emitted, indicating that the recording will start soon. The recording itself is indicated by the third characteristic sound signal. If the significant base line wandering occurs in any phase of the procedure, the procedure will be repeated from the beginning. Doing so enables generation and sending of high-quality recording of special leads, which makes possible the accurate reconstruction of standard ECG leads.

The arrangement of integrated electrodes described above, their positioning, the way of recording, and described system for eliminating the base line wandering of recorded signals minimize the errors in the reconstruction of standard ECG leads, making the accuracy of recording similar to the standard ECG devices.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted except in the spirit of the appended claims.

What is claimed is:

1. A wearable device configured to generate special ECG signals for constructing a 12-lead ECG, the special ECG signals numbering less than twelve and being combinable with a calibration matrix in order to construct the 12-lead ECG, the wearable device comprising:
a belt having one or more belt electrodes;
a waistband having one or more waistband electrodes, the belt and waistband electrodes configured to contact the skin of a wearer and obtain electrical signals therefrom; and
a host unit in electrical communication with the belt and waistband electrodes, the host unit including circuitry for generating the special ECG signals from one or more of the acquired electrical signals and circuitry for transmitting information based on the special ECG signals to a location remote from the wearable device.

2. The device of claim 1, wherein the total number of skin-contacting electrodes is four.

3. The device of claim 1, wherein the total number of skin-contacting electrodes is five.

4. The device of claim 1, wherein the belt contains three belt electrodes.

5. The device of claim 1, wherein the waistband contains two waistband electrodes.

6. The device of claim 1, wherein the host unit contains at least one host unit electrode configured to contact the skin of the wearer and obtain electrical signals therefrom.

7. The device of claim 1, wherein at least one of the belt electrodes is a common electrode.

8. The device of claim 1, wherein at least one of the belt electrodes is a ground electrode.

9. The device of claim 1, wherein at least one of the waistband electrodes is a common electrode.

10. The device of claim 1, wherein at least one of the waistband electrodes is a ground electrode.

11. The device of claim 1, wherein transmission is conducted in real time.

12. The device of claim 1, wherein transmission is conducted at prescribed time intervals.

13. The device of claim 1, wherein transmission comprises downloading the information using a dedicated connection.

14. A bedside cardiac monitoring system comprising:
a diagnostic center configured to construct a 12-lead ECG of a patient using a special ECG signals numbering less than twelve by combining the special ECG signals with a transformation matrix; and
a wearable device configured to generate the special ECG signals and including:
a belt having one or more belt electrodes;
a waistband having one or more waistband electrodes, the belt and waistband electrodes configured to contact the skin of the patient and obtain electrical signals therefrom; and
a host unit in electrical communication with the belt and waistband electrodes, the host unit including circuitry for generating the special ECG signals from one or more of the acquired electrical signals and circuitry for transmitting information based on the special ECG signals to a location remote from the wearable device.

15. The device of claim 14, wherein the total number of skin-contacting electrodes is four.

16. The system of claim 14, wherein the total number of skin-contacting electrodes is five.

17. The system of claim 14, wherein the belt contains three belt electrodes.

18. The system of claim 14, wherein the waistband contains two waistband electrodes.

19. The system of claim 14, wherein the host unit contains at least one host unit electrode configured to contact the skin of the wearer and obtain electrical signals therefrom.

20. The system of claim 14, wherein at least one of the belt electrodes is a common electrode.

21. The system of claim 14, wherein at least one of the belt electrodes is a ground electrode.

22. The system of claim 14, wherein at least one of the waistband electrodes is a common electrode.

23. The system of claim 14, wherein at least one of the waistband electrodes is a ground electrode.

24. The system of claim 14, wherein transmission is conducted in real time.

25. The system of claim 14, wherein transmission is conducted at prescribed time intervals.

26. The system of claim 14, wherein transmission comprises downloading the information using a dedicated connection.

27. A method for generating a 12-lead ECG of a patient comprising:
using belt-mounted electrodes to obtain electrical signals from the patient;
using waistband-mounted electrodes to obtain electrical signals from the patient;
using a host unit to generate special ECG signals from the electrical signals obtained from the belt-mounted and waistband-mounted electrodes, and to transmit information based on the special ECG signals to a diagnostic center, the special ECG signals numbering less than 12; and
constructing a 12-lead ECG by combining the special ECG signals with a transformation matrix.

28. The method of claim 27, wherein the total number of electrodes for obtaining electrical signals from the patient is four.

29. The method of claim 27, wherein the total number of electrodes for obtaining electrical signals from the patient is five.

30. The method of claim 27, further comprising displaying the constructed 12-lead ECG.

31. The method of claim 27, further comprising automatically monitoring the 12-lead ECG.

32. The method of claim 27, further comprising signaling an improper connection.

33. The method of claim 27, further comprising signaling a proper connection.

34. The method of claim 27, wherein transmission is conducted in real time.

35. The method of claim 27, wherein transmission is conducted at prescribed time intervals.

36. The method of claim 27, wherein transmission comprises downloading the information using a dedicated connection.

37. A wearable device configured to generate special ECG signals from at least four non-coplanar electrodes for constructing a 12-lead ECG, the special ECG signals numbering less than twelve and being combinable with a calibration matrix in order to construct the 12-lead ECG, the wearable device comprising:
at least a first wearable component having one or more electrodes configured to contact the skin of a wearer and obtain electrical signals therefrom; and
a host unit in electrical communication with the first wearable component, the host unit including circuitry for generating the special ECG signals from one or more of the acquired electrical signals and circuitry for transmitting information based on the special ECG signals to a location remote from the wearable device.

38. The device of claim 37, wherein the total number of skin-contacting electrodes is four.

39. The device of claim 37, wherein the total number of skin-contacting electrodes is five.

40. The device of claim 37, wherein the first wearable component contains three belt electrodes.

41. The device of claim 37, wherein the host unit contains at least one host unit electrode configured to contact the skin of the wearer and obtain electrical signals therefrom.

42. The device of claim 37, wherein transmission is conducted in real time.

43. The device of claim 37, wherein transmission is conducted at prescribed time intervals.

44. The device of claim 37, wherein transmission comprises downloading the information using a dedicated connection.
